(19) United States
(12) Patent Application Publication
(10) Pub. No.: US 2011/0172639 A1
(43) Pub. Date: Jul. 14, 2011

(54) DEVICE AND METHOD FOR DELIVERY OF MICRONEEDLE TO DESIRED DEPTH WITHIN THE SKIN

(75) Inventors: Benjamin J. Moga, Madison, WI (US); Kent B. Chase, Sun Prairie, WI (US); Garrick D.S. Smith, Madison, WI (US)

(73) Assignees: Ratio, Inc.; FluGen, Inc.

(21) Appl. No.: 12/684,844

(22) Filed: Jan. 8, 2010

(51) Int. Cl. A61M 5/32

(52) U.S. Cl. 604/506; 604/239; 604/180

(57) ABSTRACT

A device for delivering a drug into the skin of a subject is provided. The device includes a drug reservoir and a microneedle having a tip, a length, and a tip sharpness. The microneedle is coupled to the reservoir. The device includes a microneedle actuator coupled to the microneedle configured to drive the microneedle into the skin of the subject upon activation. The tip sharpness and the actuator allow the microneedle to pass through an outer layer of the skin upon activation, and the length is limited such that the tip does not extend past a desired depth below the surface of the skin of the subject, wherein the desired depth is located in the papillary dermis or the reticular dermis.

### BACKGROUND

The present invention relates generally to the field of drug delivery devices. The present invention relates specifically to a drug delivery device and method for delivery drug to the compliant layer of the skin.

An active agent or drug (e.g., pharmaceuticals, vaccines, hormones, nutrients, etc.) may be administered to a patient through various means. For example, a drug may be ingested, inhaled, injected, delivered intravenously, etc. In some applications, a drug may be administered transdermally. In some transdermal applications, such as transdermal nicotine or birth control patches, a drug is absorbed through the skin. Passive transdermal patches often include an absorbent layer or membrane that is placed on the outer layer of the skin. The membrane typically contains a dose of a drug that is allowed to be absorbed through the skin to deliver the substance to the patient. Typically, only drugs that are readily absorbed through the outer layer of the skin may be delivered with such devices.

Other drug delivery devices are configured to provide for increased skin permeability to the delivered drugs. For example, some devices use a structure, such as one or more microneedles, to facilitate transfer of the drug into the skin. Solid microneedles may be coated with a dry drug substance. The puncture of the skin by the solid microneedles increases permeability of the skin allowing for absorption of the drug substance. Hollow microneedles may be used to provide a fluid channel for drug delivery below the outer layer of the skin. Other active transdermal devices utilize other mechanisms (e.g., iontophoresis, sonophoresis, etc.) to increase skin permeability to facilitate drug delivery.

### SUMMARY

One embodiment of the invention relates to a device for delivering a drug into the skin of a subject. The device includes a drug reservoir and a microneedle having a tip, a length, and a tip sharpness. The microneedle is coupled to the reservoir. The device includes a microneedle actuator coupled to the microneedle configured to drive the microneedle into the skin of the subject upon activation. The tip sharpness and the actuator allow the microneedle to pass through an outer layer of the skin upon activation, and the length is limited such that the tip does not extend past a desired depth below the surface of the skin of the subject, where the desired depth is located in the papillary dermis or the reticular dermis.

Another embodiment of the invention relates to drug delivery device for delivering a liquid drug into the skin of a subject. The device includes a drug reservoir storing a dose of the liquid drug, a conduit coupled to the drug reservoir and a hollow microneedle having a tip, a length and a tip sharpness. The hollow microneedle is coupled to the conduit, and the conduit provides fluid communication between the drug reservoir and the hollow microneedle such that the drug is permitted to flow from the drug reservoir through the conduit and through the hollow microneedle to the skin of the subject. The device includes a microneedle actuator coupled to the hollow microneedle and configured to drive the hollow microneedle into the skin of the subject upon activation, and an engagement element configured to adhere to the skin of the subject such that the engagement element resists downward depression and/or deformation of the skin surface caused by the hollow microneedle during activation. At least one of the tip sharpness, the actuator and the engagement element is configured to reduce depression of the skin of the subject caused by the hollow microneedle following activation, and the microneedle length allows the tip (and/or the outlet) of the hollow microneedle to be delivered to the papillary dermis or reticular dermis of the subject.

Another embodiment of the invention relates to a method of delivering a drug to the skin of a subject. The method includes providing a drug delivery device. The drug delivery device includes a drug reservoir, a microneedle coupled to the reservoir and a microneedle actuator coupled to the microneedle configured to drive the microneedle into the skin of the subject upon activation. The microneedle includes a tip, a length and a tip sharpness. The method includes selecting at least one of the length, the tip sharpness and the microneedle actuator to allow the tip (and/or the outlet) to be delivered to a desired depth below the surface of the skin of the subject where the desired depth is located in the papillary dermis or the reticular dermis and activating the microneedle actuator to insert the microneedle to the desired depth within the skin of the subject. The method includes delivering the drug to the skin of the subject via the microneedle.

Alternative exemplary embodiments relate to other features and combinations of features as may be generally recited in the claims

### BRIEF DESCRIPTION OF THE FIGURES

This application will become more fully understood from the following detailed description, taken in conjunction with the accompanying figures, wherein like reference numerals refer to like elements in which:

FIG. 1 is a perspective view of a drug delivery device assembly having a cover and a protective membrane according to an exemplary embodiment;

FIG. 2 is a perspective view of a drug delivery device according to an exemplary embodiment after both the cover and protective membrane have been removed;

FIG. 3 is a exploded perspective view of a drug delivery device assembly according to an exemplary embodiment;

FIG. 4 is a exploded perspective view of a drug delivery device showing various components mounted within the device housing according to an exemplary embodiment;

FIG. 5 is a exploded perspective view of a drug delivery device showing various components removed from the device housing according to an exemplary embodiment;

FIG. 6 is a perspective sectional view showing a drug delivery device prior to activation according to an exemplary embodiment;

FIG. 7 is a perspective sectional view showing a drug delivery device following activation according to an exemplary embodiment;

FIG. 8 is a side sectional view showing a drug delivery device following activation according to an exemplary embodiment;

FIG. 9 is a side sectional view showing a drug delivery device following delivery of a drug according to an exemplary embodiment;

FIG. 10 is a sectional view showing a portion of a drug delivery device prior to activation according to an exemplary embodiment;

FIG. 11 is a sectional view showing a portion of a drug delivery device following activation according to an exemplary embodiment;

FIG. 12 is an enlarged sectional view of a portion of a drug delivery device following activation according to an exemplary embodiment; and

FIG. 13 is an enlarged sectional view of a microneedle of a drug delivery device following activation according to an exemplary embodiment.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

Referring generally to the figures, a substance delivery device assembly is shown according to various exemplary embodiments. The delivery device assembly includes various packaging and/or protective elements that provide for protection during storage and transportation. The assembly also includes a substance delivery device that is placed in contact with the skin of a subject (e.g., a human or animal, etc.) prior to delivery of the substance to the subject. After the device is affixed to the skin of the subject, the device is activated in order to deliver the substance to the subject. Following delivery of the substance, the device is removed from the skin.

The delivery device described herein may be utilized to deliver any substance that may be desired. In one embodiment, the substance to be delivered is a drug, and the delivery device is a drug delivery device configured to deliver the drug to a subject. As used herein the term “drug” is intended to include any substance delivered to a subject for any therapeutic, preventative or medicinal purpose (e.g., vaccines, pharmaceuticals, nutrients, nutraceuticals, etc.). In one such embodiment, the drug delivery device is a vaccine delivery device configured to deliver a dose of vaccine to a subject. In one embodiment, the delivery device is configured to deliver a flu vaccine. The embodiments discussed herein relate primarily to a device configured to deliver a substance intradermally. In other embodiments, the device may be configured to deliver a substance transdermally or may be configured to deliver drugs directly to an organ other than the skin.

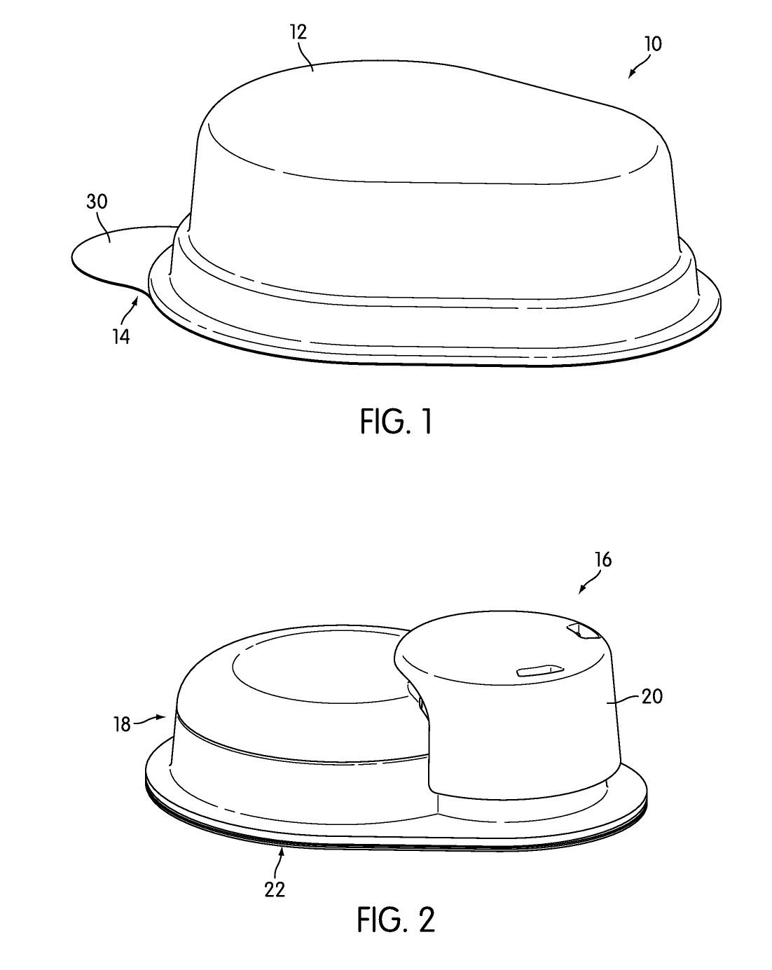

Referring to FIG. 1, drug delivery device assembly 10 is depicted according to an exemplary embodiment. Drug delivery device assembly 10 includes an outer protective cover 12 and a protective membrane or barrier 14 that provides a sterile seal for drug delivery device assembly 10. As shown in FIG. 1, drug delivery device assembly 10 is shown with cover 12 and protective barrier 14 in an assembled configuration. Generally, cover 12 and protective barrier 14 protect various components of drug delivery device 16 during storage and transport prior to use by the end user. In various embodiments, cover 12 may be made of a relatively rigid material (e.g., plastic, metal, cardboard, etc.) suitable to protect other components of drug delivery device assembly 10 during storage or shipment. As shown, cover 12 is made from a non-transparent material. However, in other embodiments cover 12 is a transparent or semi-transparent material.

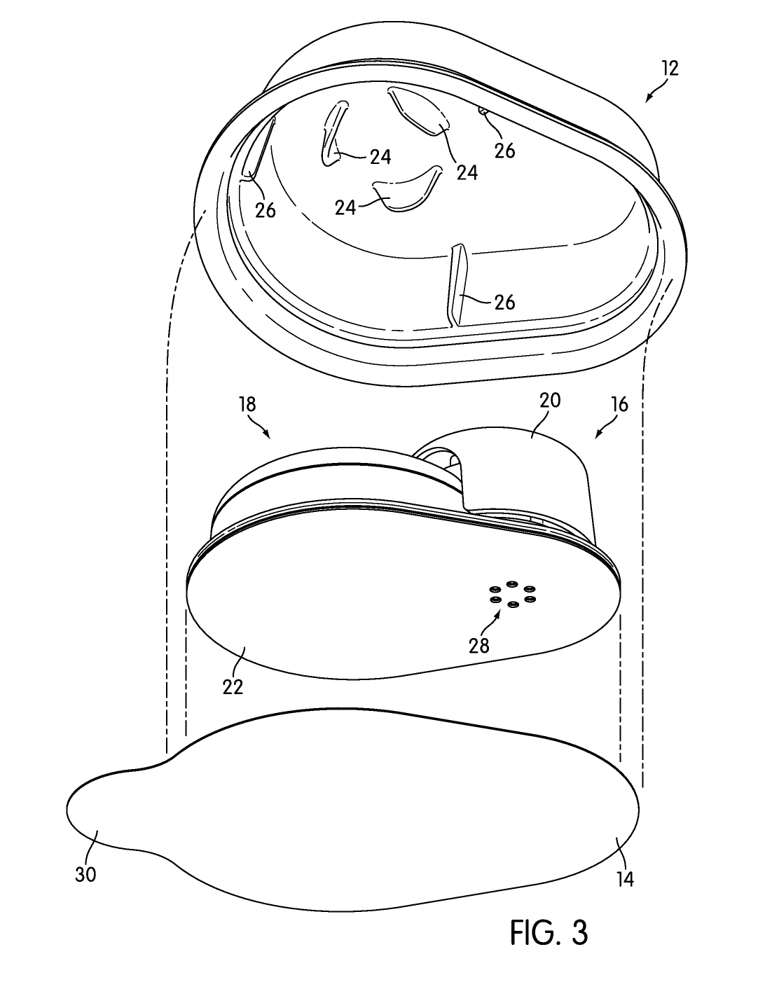

As shown in FIG. 2 and FIG. 3, the drug delivery device assembly includes delivery device 16. Delivery device 16 includes a housing 18, an activation control, shown as, but not limited to, button 20, and an attachment element, shown as, but not limited to, adhesive layer 22. Adhesive layer 22 includes one or more holes 28 (see FIG. 3). Holes 28 provide a passageway for one or more hollow drug delivery microneedles as discussed in more detail below. During storage and transport, cover 12 is mounted to housing 18 of delivery device 16 such that delivery device 16 is received within cover 12. In the embodiment shown, cover 12 includes three projections or tabs 24 extending from the inner surface of the top wall of cover 12 and three projections or tabs 26 extending from the inner surface of the sidewall of cover 12. When cover 12 is mounted to delivery device 16, tabs 24 and 26 contact the outer surface of housing 18 such that delivery device 16 is positioned properly and held within cover 12. Protective barrier 14 is attached to the lower portion of cover 12 covering adhesive layer 22 and holes 28 during storage and shipment. Together, cover 12 and protective barrier 14 act to provide a sterile and hermetically sealed packaging for delivery device 16.

Referring to FIG. 3, to use delivery device 16 to deliver a drug to a subject, protective barrier 14 is removed exposing adhesive layer 22. In the embodiment shown, protective barrier 14 includes a tab 30 that facilitates griping of protective barrier 14 during removal. Once adhesive layer 22 is exposed, delivery device 16 is placed on the skin. Adhesive layer 22 is made from an adhesive material that forms a nonpermanent bond with the skin of sufficient strength to hold delivery device 16 in place on the skin of the subject during use. Cover 12 is released from delivery device 16 exposing housing 18 and button 20 by squeezing the sides of cover 12. With delivery device 16 adhered to the skin of the subject, button 20 is pressed to trigger delivery of the drug to the patient. When delivery of the drug is complete, delivery device 16 may be detached from the skin of the subject by applying sufficient force to overcome the grip generated by adhesive layer 22.

In one embodiment, delivery device 16 is sized to be conveniently wearable by the user during drug delivery. In one embodiment, the length of delivery device 16 along the device's long axis is 53.3 mm, the length of delivery device 16 along the device's short axis (at its widest dimension) is 48 mm, and the height of delivery device 16 at button 20 following activation is 14.7 mm. However, in other embodiments other dimensions are suitable for a wearable drug delivery device. For example, in another embodiment, the length of delivery device 16 along the device's long axis is between 40 mm and 80 mm, the length of delivery device 16 along the device's short axis (at its widest dimension) is between 30 mm and 60 mm, and the height of delivery device 16 at button 20 following activation is between 5 mm and 30 mm. In another embodiment, the length of delivery device 16 along the device's long axis is between 50 mm and 55 mm, the length of delivery device 16 along the device's short axis (at its widest dimension) is between 45 mm and 50 mm, and the height of delivery device 16 at button 20 following activation is between 10 mm and 20 mm.

While in the embodiments shown the attachment element is shown as, but not limited to, adhesive layer 22, other attachment elements may be used. For example, in one embodiment, delivery device 16 may be attached via an elastic strap. In another embodiment, delivery device 16 may not include an attachment element and may be manually held in place during delivery of the drug. Further, while the activation control is shown as button 20, the activation control may be a switch, trigger, or other similar element, or may be more than one button, switch, trigger, etc., that allows the user to trigger delivery of the drug.

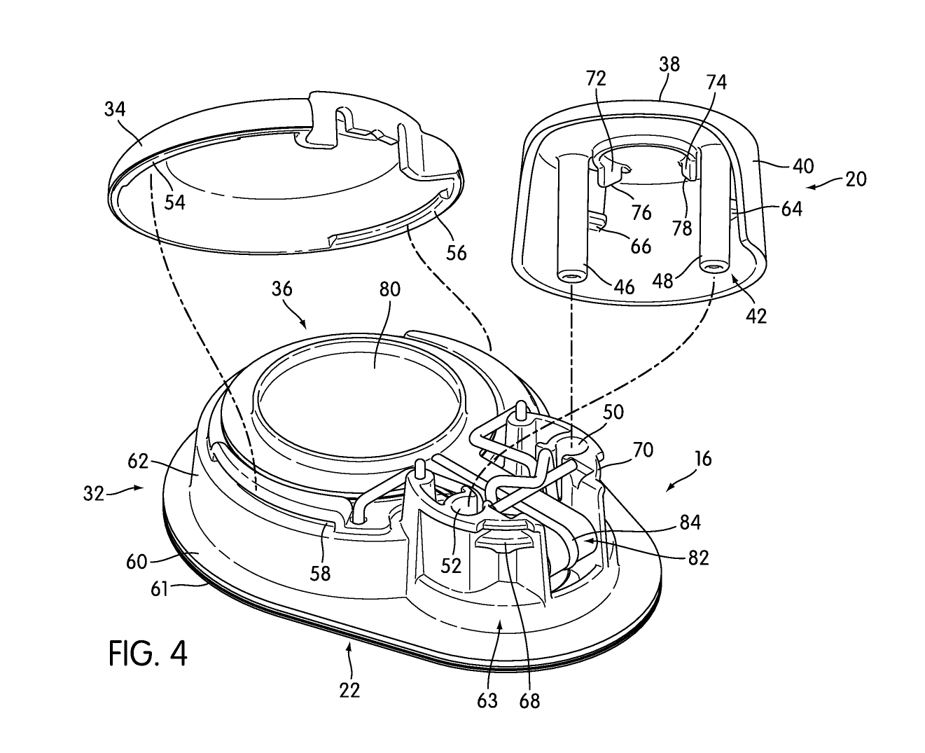

Referring to FIG. 4, housing 18 of delivery device 16 includes a base portion 32 and a reservoir cover 34. Base portion 32 includes a flange 60, a bottom tensile member, shown as bottom wall 61, a first support portion 62 and a second support portion 63. In the embodiment shown, bottom wall 61 is a rigid wall that is positioned below flange 60. As shown in FIG. 4, the outer surface of first support portion 62 is generally cylindrically shaped and extends upward from flange 60. Second support portion 63 is generally cylindrically shaped and extends upward from flange 60 to a height above first support portion 62. As shown in FIG. 4, delivery device 16 includes a substance delivery assembly 36 mounted within base portion 32 of housing 18.

Reservoir cover 34 includes a pair of tabs 54 and 56 that each extend inwardly from a portion of the inner edge of cover 34. Base portion 32 includes a recess 58 and second recess similar to recess 58 on the opposite side of base portion 32. As shown in FIG. 4, both recess 58 and the opposing recess are formed in the upper peripheral edge of the outer surface of first support portion 62. When reservoir cover 34 is mounted to base portion 32, tab 54 is received within recess 58 and tab 56 is received within the similar recess on the other side of base portion 32 to hold cover 34 to base portion 32.

As shown in FIG. 4, button 20 includes a top wall 38. Button 20 also includes a sidewall or skirt 40 that extends from a portion of the peripheral edge of top wall 38 such that skirt 40 defines an open segment 42. Button 20 is shaped to receive the generally cylindrical shaped second support portion 63 of base portion 32. Button 20 includes a first mounting post 46 and a second mounting post 48 both extending in a generally perpendicular direction from the lower surface of top wall 38. Second support portion 63 includes a first channel 50 and a second channel 52. Mounting posts 46 and 48 are slidably received within channels 50 and 52, respectively, when button 20 is mounted to second support portion 63. Mounting posts 46 and 48 and channels 50 and 52 act as a vertical movement guide for button 20 to help ensure that button 20 moves in a generally downward vertical direction in response to a downward force applied to top wall 38 during activation of delivery device 16. Precise downward movement of button 20 ensures button 20 interacts as intended with the necessary components of substance delivery assembly 36 during activation.

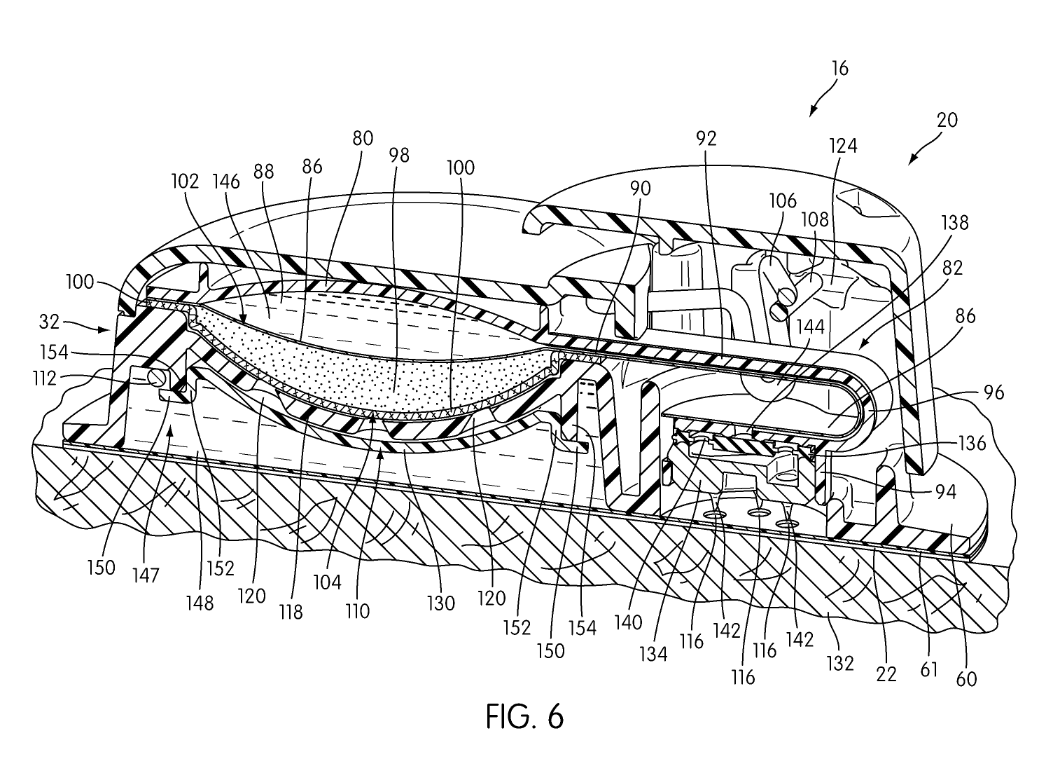

Button 20 also includes a first support ledge 64 and a second support ledge 66 both extending generally perpendicular to the inner surface of sidewall 40. The outer surface of second support portion 63 includes a first button support surface 68 and second button support surface 70. When button 20 is mounted to second support portion 63, first support ledge 64 engages and is supported by first button support surface 68 and second support ledge 66 engages and is supported by second button support surface 70. The engagement between ledge 64 and surface 68 and between ledge 66 and surface 70 supports button 20 in the pre-activation position (shown for example in FIG. 6). Button 20 also includes a first latch engagement element 72 and a second latch engagement element 74 both extending in a generally perpendicular direction from the lower surface of top wall 38. First latch engagement element 72 includes an angled engagement surface 76 and second latch engagement element 74 includes an angled engagement surface 78.

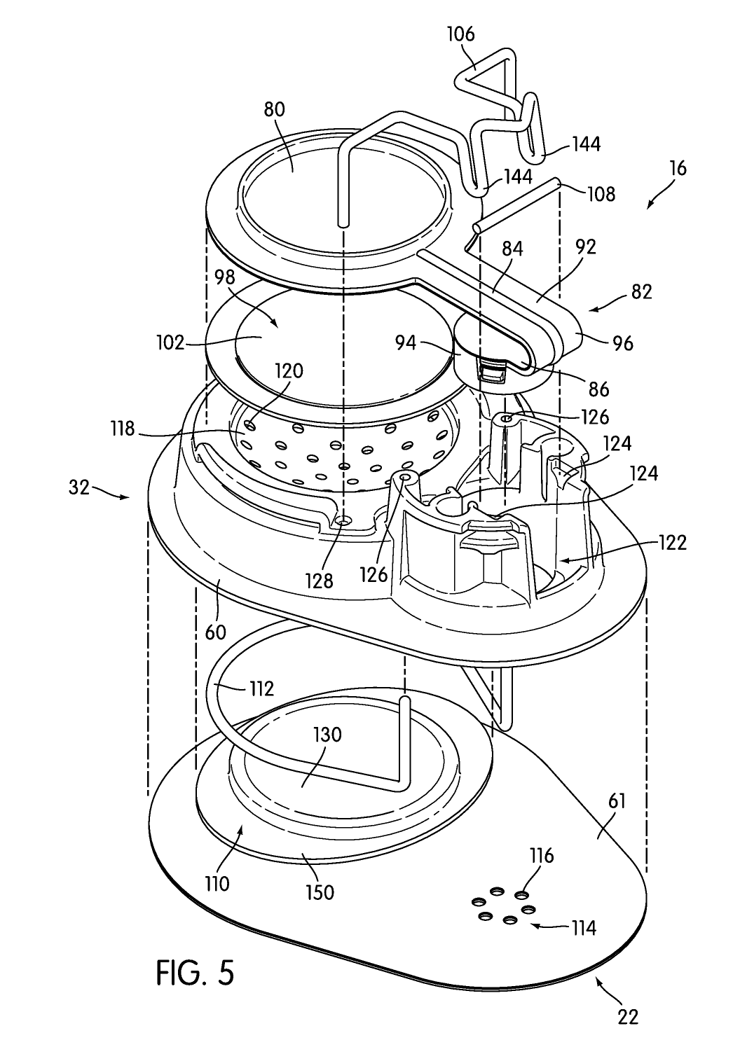

Referring to FIG. 4 and FIG. 5, substance delivery assembly 36 includes a drug reservoir base 80 and drug channel arm 82. The lower surface of drug channel arm 82 includes a depression or groove 84 that extends from reservoir base 80 along the length of drug channel arm 82. As shown in FIG. 4 and FIG. 5, groove 84 appears as a rib protruding from the upper surface of drug channel arm 82. Substance delivery assembly 36 further includes a flexible barrier film 86 adhered to the inner surfaces of both drug reservoir base 80 and drug channel arm 82. Barrier film 86 is adhered to form a fluid tight seal or a hermetic seal with drug reservoir base 80 and channel arm 82. In this arrangement (shown best in FIGS. 6-9), the inner surface of drug reservoir base 80 and the inner surface of barrier film 86 form a drug reservoir 88, and the inner surface of groove 84 and the inner surface of barrier film 86 form a fluid channel, shown as, but not limited to, drug channel 90. In this embodiment, drug channel arm 82 acts as a conduit to allow fluid to flow from drug reservoir 88. As shown, drug channel arm 82 includes a first portion 92 extending from drug reservoir base 80, a microneedle attachment portion, shown as, but not limited to, cup portion 94, and a generally U-shaped portion 96 joining the first portion 92 to the cup portion 94. In the embodiment shown, drug reservoir base 80 and drug channel arm 82 are made from an integral piece of polypropylene. However, in other embodiments, drug reservoir base 80 and drug channel arm 82 may be separate pieces joined together and may be made from other plastics or other materials.

Substance delivery assembly 36 includes a reservoir actuator or force generating element, shown as, but not limited to, hydrogel 98, and a fluid distribution element, shown as, but not limited to, wick 100 in FIG. 6. Because FIG. 5 depicts delivery device 16 in the pre-activated position, hydrogel 98 is formed as a hydrogel disc and includes a concave upper surface 102 and a convex lower surface 104. As shown, wick 100 is positioned below hydrogel 98 and is shaped to generally conform to the convex shape of lower surface 104.

Substance delivery assembly 36 includes a microneedle activation element or microneedle actuator, shown as, but not limited to, torsion rod 106, and a latch element, shown as, but not limited to, latch bar 108. As explained in greater detail below, torsion rod 106 stores energy, which upon activation of delivery device 16, is transferred to one or more microneedles causing the microneedles to penetrate the skin. Substance delivery assembly 36 also includes a fluid reservoir plug 110 and plug disengagement bar 112. Bottom wall 61 is shown removed from base portion 32, and adhesive layer 22 is shown coupled to the lower surface of bottom wall 61. Bottom wall 61 includes one or more holes 114 that are sized and positioned to align with holes 28 in adhesive layer 22. In this manner, holes 114 in bottom wall 61 and holes 28 in adhesive layer 22 form channels, shown as needle channels 116.

As shown in FIG. 5, first support portion 62 includes a support wall 118 that includes a plurality of fluid channels 120. When assembled, wick 100 and hydrogel 98 are positioned on support wall 118 below drug reservoir 88. As shown, support wall 118 includes an upper concave surface that generally conforms to the convex lower surfaces of wick 100 and hydrogel 98. Fluid reservoir plug 110 includes a concave central portion 130 that is shaped to generally conform to the convex lower surface of support wall 118. First support portion 62 also includes a pair of channels 128 that receive the downwardly extending segments of torsion rod 106 such that the downwardly extending segments of torsion rod 106 bear against the upper surface of bottom wall 61 when delivery device 16 is assembled. Second support portion 63 includes a central cavity 122 that receives cup portion 94, U-shaped portion 96 and a portion of first portion 92 of drug channel arm 82. Second support portion 63 also includes a pair of horizontal support surfaces 124 that support latch bar 108 and a pair of channels 126 that slidably receive the vertically oriented portions of plug disengagement bar 112.

Referring to FIG. 6, a perspective, sectional view of delivery device 16 is shown attached or adhered to skin 132 of a subject prior to activation of the device. As shown, adhesive layer 22 provides for gross attachment of the device to skin 132 of the subject. Delivery device 16 includes a microneedle component, shown as, but not limited to, microneedle array 134, having a plurality of microneedles, shown as, but not limited to, hollow microneedles 142, extending from the lower surface of microneedle array 134. In the embodiment shown, microneedle array 134 includes an internal channel 141 allowing fluid communication from the upper surface of microneedle array 134 to the tips of hollow microneedles 142. Delivery device 16 also includes a valve component, shown as, but not limited to, check valve 136. Both microneedle array 134 and check valve 136 are mounted within cup portion 94. Drug channel 90 terminates in an aperture or hole 138 positioned above check valve 136. In the pre-activation or inactive position shown in FIG. 6, check valve 136 blocks hole 138 at the end of drug channel 90 preventing a substance, shown as, but not limited to, drug 146, within drug reservoir 88 from flowing into microneedle array 134. While the embodiments discussed herein relate to a drug delivery device that utilizes hollow microneedles, in other various embodiments, other microneedles, such as solid microneedles, may be utilized.

As shown in FIG. 6, in the pre-activation position, latch bar 108 is supported by horizontal support surfaces 124. Latch bar 108 in turn supports torsion rod 106 and holds torsion rod 106 in the torqued, energy storage position shown in FIG. 6. Torsion rod 106 includes a U-shaped contact portion 144 that bears against a portion of the upper surface of barrier film 86 located above cup portion 94. In another embodiment, U-shaped contact portion 144 is spaced above barrier film 86 (i.e., not in contact with barrier film 86) in the pre-activated position.

Delivery device 16 includes an activation fluid reservoir, shown as, but not limited to, fluid reservoir 147, that contains an activation fluid, shown as, but not limited to, water 148. In the embodiment shown, fluid reservoir 147 is positioned generally below hydrogel 98. In the pre-activation position of FIG. 6, fluid reservoir plug 110 acts as a plug to prevent water 148 from flowing from fluid reservoir 147 to hydrogel 98. In the embodiment show, reservoir plug 110 includes a generally horizontally positioned flange 150 that extends around the periphery of plug 110. Reservoir plug 110 also includes a sealing segment 152 that extends generally perpendicular to and vertically away from flange 150. Sealing segment 152 of plug 110 extends between and joins flange 150 with the concave central portion 130 of plug 110. The inner surface of base portion 32 includes a downwardly extending annular sealing segment 154. The outer surfaces of sealing segment 152 and/or a portion of flange 150 abut or engage the inner surface of annular sealing segment 154 to form a fluid-tight seal preventing water from flowing from fluid reservoir 147 to hydrogel 98 prior to device activation.

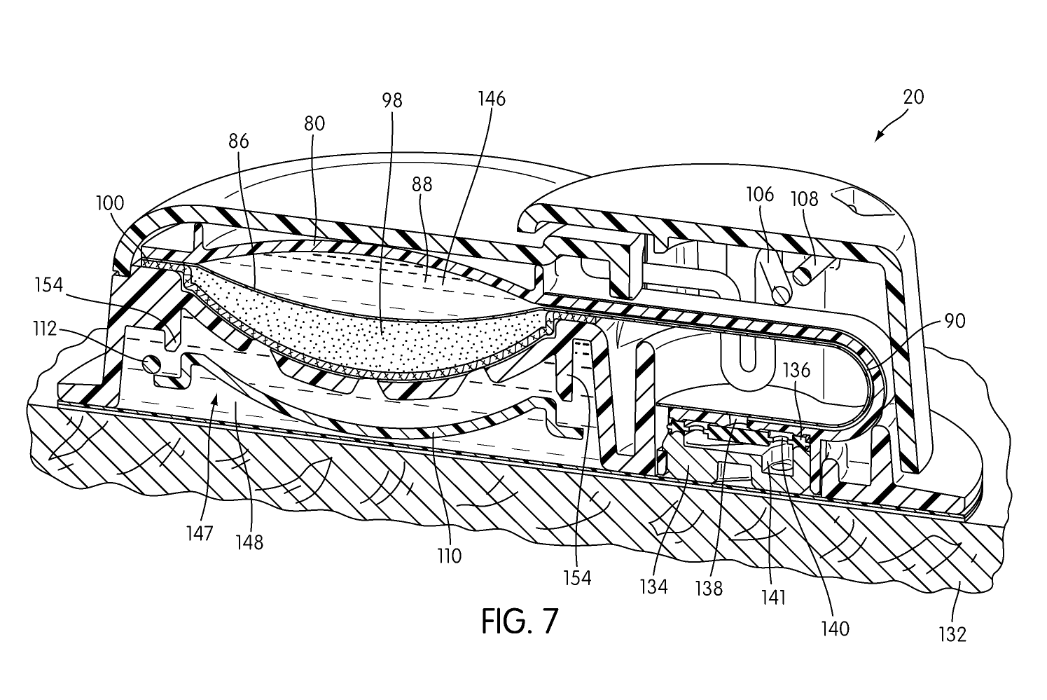

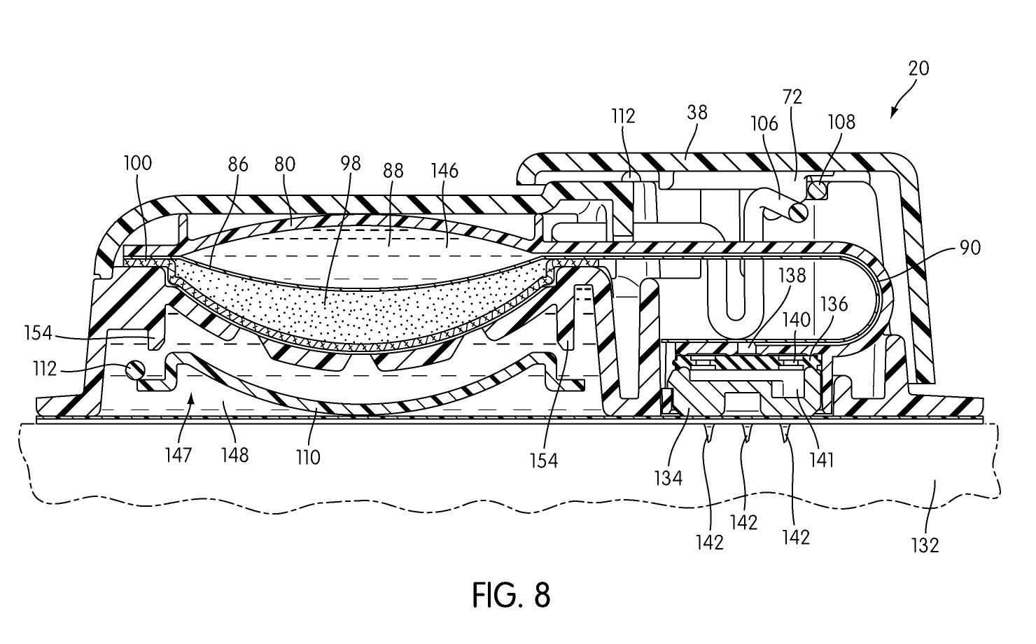

Referring to FIG. 7 and FIG. 8, delivery device 16 is shown immediately following activation. In FIG. 8, skin 132 is drawn in broken lines to show hollow microneedles 142 after insertion into the skin of the subject. To activate delivery device 16, button 20 is pressed in a downward direction (toward the skin). Movement of button 20 from the pre-activation position of FIG. 6 to the activated position causes activation of both microneedle array 134 and of hydrogel 98. Depressing button 20 causes first latch engagement element 72 and second latch engagement element 74 to engage latch bar 108 and to force latch bar 108 to move from beneath torsion rod 106 allowing torsion rod 106 to rotate from the torqued position of FIG. 6 to the seated position of FIG. 7. The rotation of torsion rod 106 drives microneedle array 134 downward and causes hollow microneedles 142 to pierce skin 132. In addition, depressing button 20 causes the lower surface of button top wall 38 to engage plug disengagement bar 112 forcing plug disengagement bar 112 to move downward. As plug disengagement bar 112 is moved downward, fluid reservoir plug 110 is moved downward breaking the seal between annular sealing segment 154 of base portion 32 and sealing segment 152 of reservoir plug 110.

With the seal broken, water 148 within reservoir 147 is put into fluid communication with hydrogel 98. As water 148 is absorbed by hydrogel 98, hydrogel 98 expands pushing barrier film 86 upward toward drug reservoir base 80. As barrier film 86 is pushed upward by the expansion of hydrogel 98, pressure within drug reservoir 88 and drug channel 90 increases. When the fluid pressure within drug reservoir 88 and drug channel 90 reaches a threshold, check valve 136 is forced open allowing drug 146 within drug reservoir 88 to flow through aperture 138 at the end of drug channel 90. As shown, check valve 136 includes a plurality of holes 140, and microneedle array 134 includes a plurality of hollow microneedles 142. Drug channel 90, hole 138, plurality of holes 140 of check valve 136, internal channel 141 of microneedle array 134 and hollow microneedles 142 define a fluid channel between drug reservoir 88 and the subject when check valve 136 is opened. Thus, drug 146 is delivered from reservoir 88 through drug channel 90 and out of the holes in the tips of hollow microneedles 142 to the skin of the subject by the pressure generated by the expansion of hydrogel 98.

In the embodiment shown, check valve 136 is a segment of flexible material (e.g., medical grade silicon) that flexes away from aperture 138 when the fluid pressure within drug channel 90 reaches a threshold placing drug channel 90 in fluid communication with hollow microneedles 142. In one embodiment, the pressure threshold needed to open check valve 136 is about 0.5-1.0 pounds per squire inch (psi). In various other embodiments, check valve 136 may be a rupture valve, a swing check valve, a ball check valve, or other type of valve the allows fluid to flow in one direction. In the embodiment shown, the microneedle actuator is a torsion rod 106 that stores energy for activation of the microneedle array until the activation control, shown as button 20, is pressed. In other embodiments, other energy storage or force generating components may be used to activate the microneedle component. For example, in various embodiments, the microneedle activation element may be a coiled compression spring or a leaf spring. In other embodiments, the microneedle component may be activated by a piston moved by compressed air or fluid. Further, in yet another embodiment, the microneedle activation element may be an electromechanical element, such as a motor, operative to push the microneedle component into the skin of the patient.

In the embodiment shown, the actuator that provides the pumping action for drug 146 is a hydrogel 98 that expands when allowed to absorb water 148. In other embodiments, hydrogel 98 may be an expandable substance that expands in response to other substances or to changes in condition (e.g., heating, cooling, pH, etc.). Further, the particular type of hydrogel utilized may be selected to control the delivery parameters. In various other embodiments, the actuator may be any other component suitable for generating pressure within a drug reservoir to pump a drug in the skin of a subject. In one exemplary embodiment, the actuator may be a spring or plurality of springs that when released push on barrier film 86 to generate the pumping action. In another embodiment, the actuator may be a manual pump (i.e., a user manually applies a force to generate the pumping action). In yet another embodiment, the actuator may be an electronic pump.

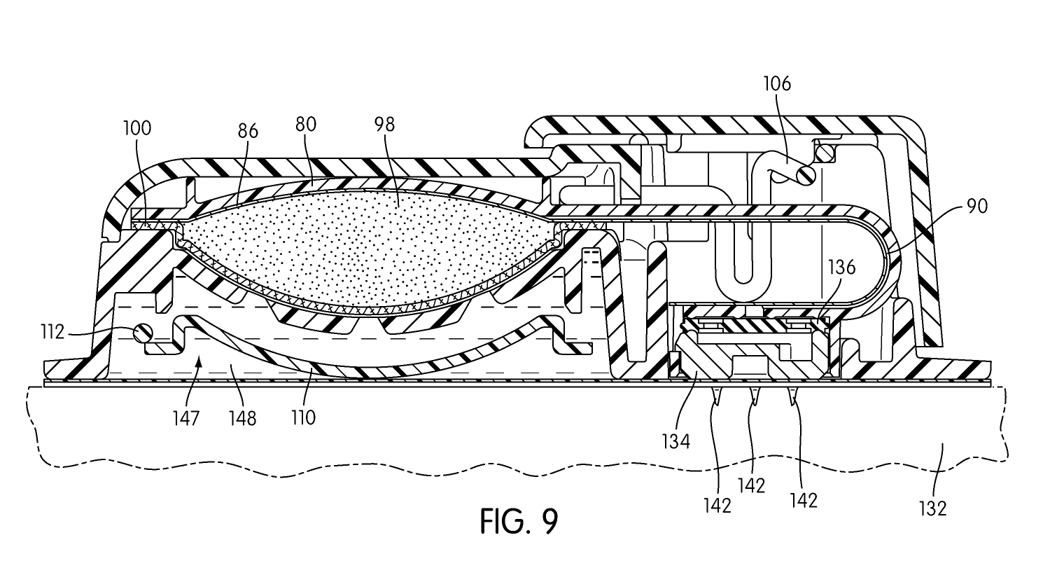

Referring to FIG. 9, delivery device 16 is shown following completion of delivery of drug 146 to the subject. In FIG. 9, skin 132 is drawn in broken lines. As shown in FIG. 9, hydrogel 98 expands until barrier film 86 is pressed against the lower surface of reservoir base 80. When hydrogel 98 has completed expansion, substantially all of drug 146 has been pushed from drug reservoir 88 into drug channel 90 and delivered to skin 132 of the subject. The volume of drug 146 remaining within delivery device 16 (i.e., the dead volume) following complete expansion by hydrogel 98 is minimized by configuring the shape of drug reservoir 88 to enable complete evacuation of the drug reservoir and by minimizing the volume of fluid pathway formed by drug channel 90, hole 138, plurality of holes 140 of check valve 136 and hollow microneedles 142. In the embodiment shown, delivery device 16 is a single-use, disposable device that is detached from skin 132 of the subject and is discarded when drug delivery is complete. However, in other embodiments, delivery device 16 may be reusable and is configured to be refilled with new drug, to have the hydrogel replaced, and/or to have the microneedles replaced.

In one embodiment, delivery device 16 and reservoir 88 are sized to deliver a dose of drug of up to approximately 500 microliters. In other embodiments, delivery device 16 and reservoir 88 are sized to allow delivery of other volumes of drug (e.g., up to 200 microliters, up to 400 microliters, up to 1 milliliter, etc.).

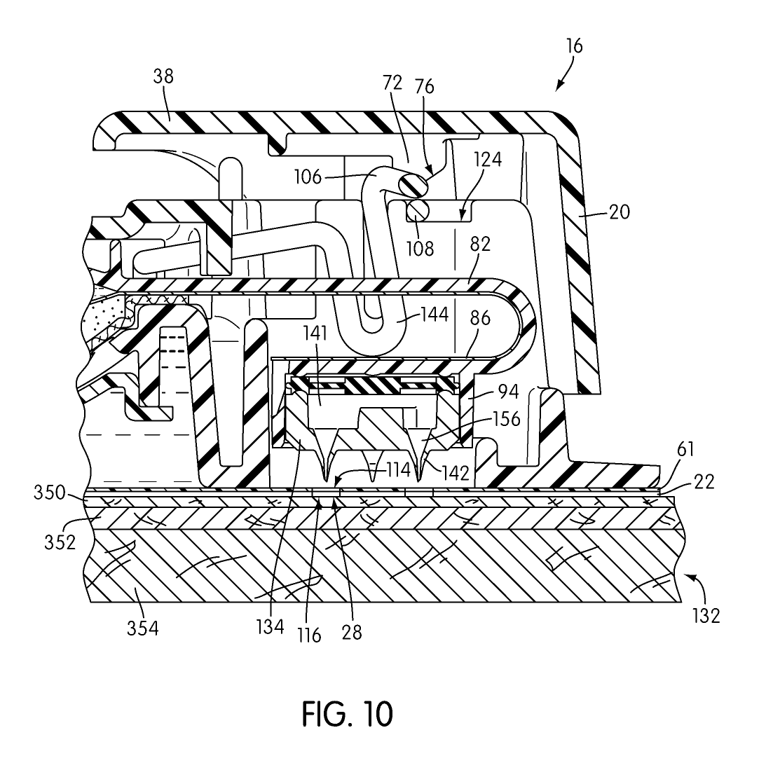

Referring generally to FIGS. 10-13, a drug delivery device, such as delivery device 16, is configured to deliver a tip and/or outlet of a microneedle to a particular predetermined or desired depth within the skin of the subject. In one embodiment, the drug delivery device may be configured to deliver a drug to a desired layer or layers of the subject's skin via the microneedle. In various embodiments, components of the drug delivery device are selected, tuned, configured, etc., such that one or more microneedles penetrate the skin of the subject such that tip of the microneedle comes to rest at a desired depth or distance within in the skin of the subject. The desired depth of microneedle penetration may depend on various factors, including the type of drug being delivered, the properties (e.g., viscosity, pH, etc.) of the drug solution, the area on the body to which the drug is being delivered, the type of microneedles used, etc. With the tip of the microneedle delivered to a desired depth, a drug may be delivered via the outlet in the tip of the microneedle.

Referring to FIG. 10, one embodiment of drug delivery device 16 configured to deliver a tip and/or the outlet of a microneedle to a desired layer of the skin is shown. Adhesive layer 22 forms a nonpermanent bond with the outer surface of skin 132 to attach drug delivery device 16 to skin 132. As shown in FIG. 10, skin 132 has three layers, an upper layer 350, a middle layer 352, and a lower layer 354. In one embodiment, upper layer 350 is the epidermis, middle layer 352 is the papillary dermis, and lower layer 354 is the reticular dermis. It should be understood the three layers of skin 132 are shown for illustrative purposes only and that while one specific embodiment discussed herein relates to delivering a microneedle tip or outlet to the papillary dermis or reticular dermis, in other embodiments drug delivery device 16 may be configured to deliver a microneedle to other layers of the skin or to other depths.

FIG. 10 shows drug delivery device 16 in the pre-activated or inactive position. Delivery device 16 includes a microneedle activation element or microneedle actuator, shown as, but not limited to, torsion rod 106. Torsion rod 106 is supported by a latch element, shown as, latch bar 108. Latch bar 108 is supported by horizontal support surface 124. In the pre-activated position, latch bar 108 engages and supports torsion rod 106. In the inactive position, first latch engagement element 72 extends from the lower surface of top wall 38 of button 20. U-shaped contact portion 144 of torsion bar 106 is in contact with barrier film 86 and is poised above microneedle array 134. In another embodiment, U-shaped contact portion 144 is spaced above barrier film 86 (i.e., not in contact with barrier film 86) in the pre-activated position. Microneedle array 134 is mounted within cup portion 94 of drug channel arm 82. In the embodiment shown, drug channel arm 82 is rigid enough to support or hold microneedle array 134 above bottom wall 61 in the inactive position.

Microneedle array 134 includes one or more microneedles 142. In the embodiment shown, microneedles 142 are cannulated, defining a central channel 156 that places the tip of each microneedle 142 in fluid communication with internal channel 141 of microneedle array 134. As shown, holes 114 in bottom wall 61 and holes 28 in adhesive layer 22 form a plurality of channels 116. In the inactive position, each microneedle 142 is poised above and aligns with one of the channels 116.

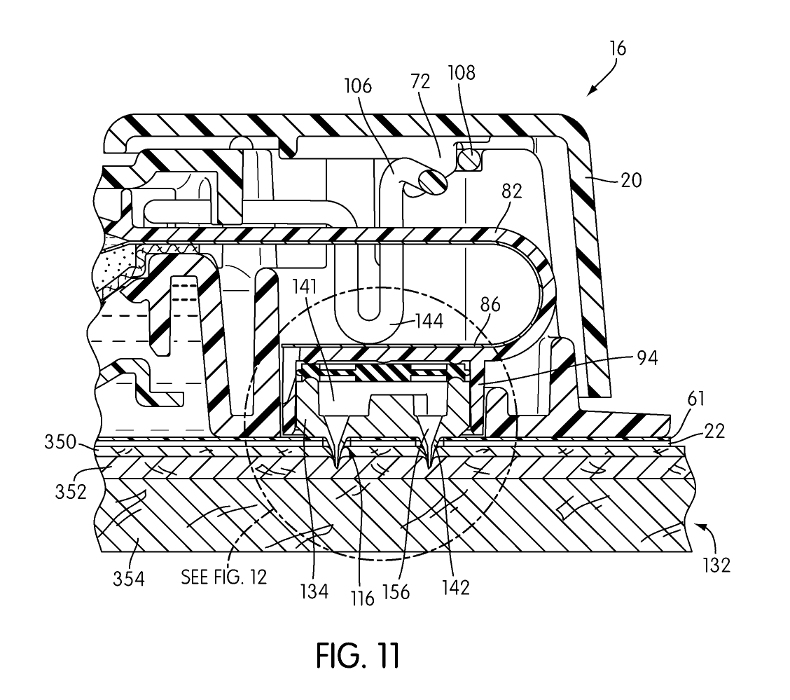

Referring to FIG. 11, delivery device 16 is shown following activation. To activate delivery device 16, a downward force is applied to button 20. As button 20 moves downward, angled engagement surface 76 of first latch engagement element 72 engages latch bar 108. As first latch engagement element 72 moves downward, latch bar 108 is pushed to the right along horizontal support surface 124 such that torsion rod 106 is released. When released, torsion rod 106 twists clockwise such that contact portion 144 moves generally downward (in the view of FIG. 11), bearing against the upper surface of barrier film 86 above microneedle array 134. The release of the energy stored in torsion rod 106 forces microneedle array 134 downward. Torsion rod 106 stores energy that is released upon depression of button 20. In this embodiment, the energy used to move microneedle array 134 from the inactive to the active position is stored by torsion rod 106 completely within housing 18.

As torsion rod 106 begins to twist clockwise, microneedle array 134 moves downward causing each microneedle 142 to move downward through channels 116 bringing the tips of microneedles 142 into contact with the upper surface of skin 132. As torsion rod 106 continues to twist clockwise, microneedles 142 pierce skin 132 of the subject. Following activation of microneedle array 134, microneedle array 134 rests against the upper surface of bottom wall 61, and microneedles 142 extend through channels 116 and are delivered to a desired depth within skin 132.

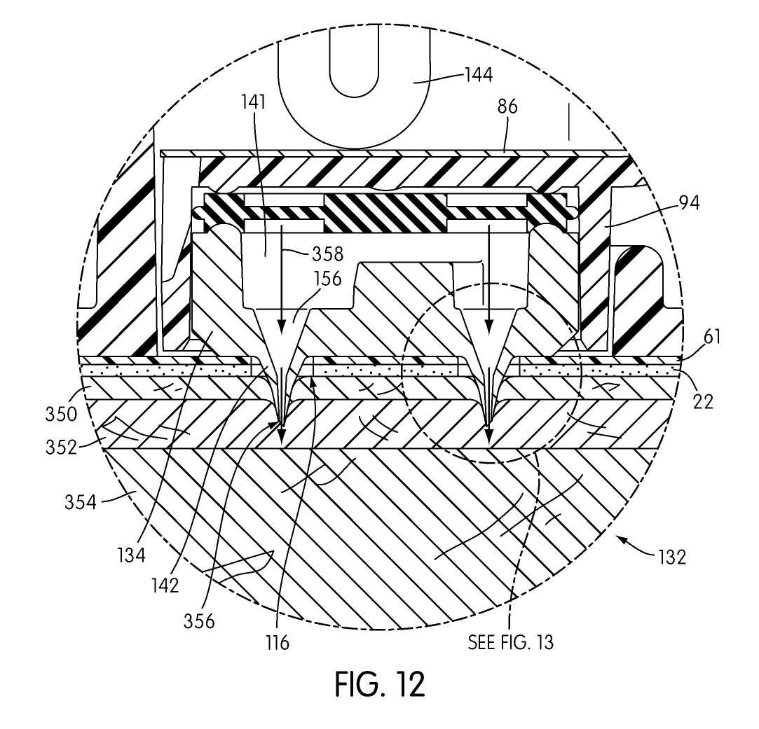

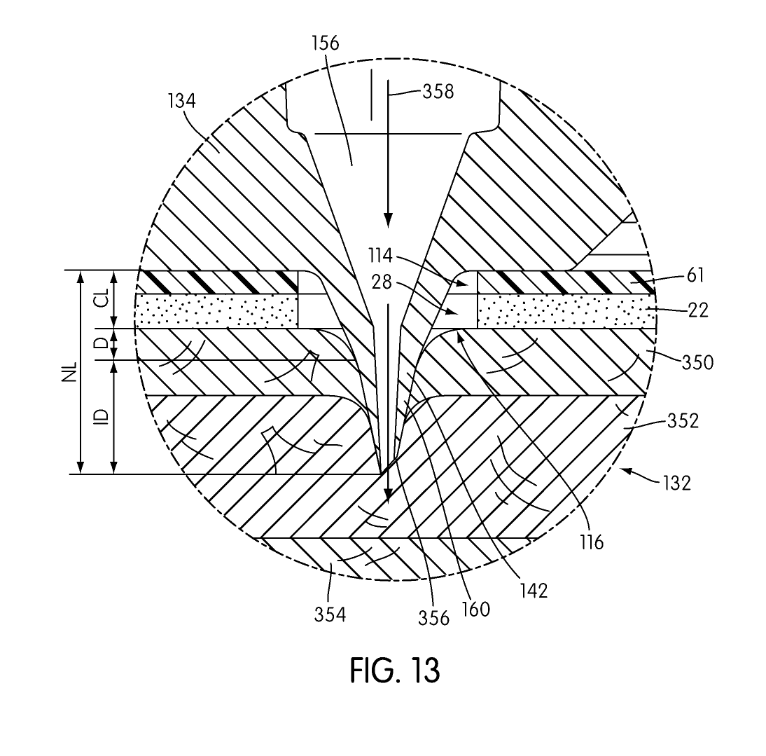

Referring to FIGS. 12 and 13, microneedles 142 are shown following activation with microneedles 142 extending to a desired depth below the outer surface of the skin. As shown in FIGS. 12 and 13, microneedles 142 have penetrated the skin such that tips 356 are positioned within middle layer 352 of skin 132. With tips 356 positioned within middle layer 352 of skin 132, drug is delivered through tips 356 of microneedles 142 into middle layer 352 of skin 132 via pressure generated by the expansion of hydrogel 98 (see FIG. 9). Flow of the drug is represented in FIGS. 12 and 13 by arrows 358.

In one embodiment, middle layer 352 is the papillary dermis and tips 356 of microneedles 142 are delivered to the papillary dermis. In this embodiment, drug is delivered via microneedles 142 to the papillary dermis layer. The papillary dermis is believed to be more compliant than either the epidermis, represented as layer 350, or the reticular dermis, represented as layer 354. Due to the compliant nature of the papillary dermis, delivery of tips 356 of microneedles 142 to the papillary dermis may be advantageous for transdermal drug delivery. When compared to the less compliant epidermis or reticular dermis, it is believed that delivery of a drug via a microneedle to the papillary dermis may allow for a greater volume of drug to be delivered via the microneedle or for a higher drug delivery rate through the microneedle because the compliant nature of the papillary dermis allows the tissue to expand and deform as the drug is delivered. Further, it is believed that delivery of drug to the papillary dermis reduces leakage of the drug back to the surface of skin 132 during drug delivery because of the compliant nature of the papillary dermis. In one embodiment, delivery device 16 is configured to deliver tip 356 of microneedle 142 to the papillary dermis of the upper arm. In another embodiment, delivery device 16 is configured to deliver tip 356 of microneedle 142 to the papillary dermis of the thigh.

In another embodiment, middle layer 352 may be the reticular dermis and tips 356 of microneedles 142 are delivered to the reticular dermis. In one particular embodiment, tips 356 may be delivered to the upper half of the reticular dermis. Tips 356 of microneedles 142 may be delivered to the reticular dermis for applications in which delivery of drug to the reticular dermis is desired. In some embodiments, with tips 356 located in the reticular dermis, delivered drug may flow upward through the skin from tips 356. This allows the drug to be delivered to both the reticular dermis and the papillary dermis. In various embodiments, tips 356 may be delivered to various depths below the outer surface of the skin. For example, in one embodiment, tips 356 may be delivered to a depth of approximately 100 micrometers to 2 millimeters below the outer surface of the skin (e.g. the skin of the upper arm). In another embodiment, tips 356 may be delivered to a depth of approximately 100 micrometers to 1.9 millimeters below the outer surface of the skin (e.g., the skin of the abdomen). In another embodiment, tips 356 may be delivered to a depth of approximately 100 micrometers to 1.1 millimeters below the outer surface of the skin. In another embodiment, tips 356 may be delivered to a depth of approximately 250 micrometers to 950 micrometers below the outer surface of the skin. In other embodiments, tips 356 may be delivered to other depth ranges (e.g., 150 micrometers to 650 micrometers, 150 micrometers to 200 micrometers, 300 micrometers to 1.25 millimeters, etc.).

Several components of drug delivery device 16 relate to the depth of delivery of tip 356 of microneedle 142. Appropriately selecting components with particular features, properties, etc., allows one to configure delivery device 16 to deliver tip 356 of microneedle 142 to a desired depth within skin 132. Generally, the delivery depth of tip 356 depends on the length of the microneedles, the sharpness of the microneedles, the force imparted to the microneedles to penetrate the skin, the length of the channels through which the microneedles extend and the amount of depression experienced by the skin following needle penetration. The delivery depth of tip 356 also varies with the number of microneedles present on microneedle array 134.

Referring to FIG. 13, microneedle 142 has a needle length NL. Channel 116 has a channel length CL. In addition, when a microneedle is brought into contact with the skin of a subject, the skin typically will depress or deform prior to puncture of the skin, and the skin may remain depressed following puncture resulting in a decrease in the effective depth within the skin that the microneedle reaches. As shown in FIG. 13, following puncture by microneedles 142, skin 132 remains depressed somewhat shown by the depth of depression D. Thus, as shown in FIG. 13 the delivery or insertion depth (relative to the top of the skin 132 at the puncture point) of microneedle 142 is shown as the distance ID. As shown in FIG. 13, the delivery depth ID equals the needle length NL minus the channel length CL minus the depression depth D.

Needle length, NL, sets the maximum potential delivery depth. As shown in FIG. 13, channel length, CL, limits the maximum delivery depth for a microneedle of a given needle length, NL. Thus, to deliver tip 356 to a desired depth, a needle length greater than the desired depth should be selected. Further, as shown in FIG. 13, channel length is a function of the thickness of both bottom wall 61 and adhesive layer 22. In one embodiment, channel length is minimized by making bottom wall 61 as thin as possible while still providing the necessary support for the components of delivery device 16 and by making adhesive layer 22 as thin as possible while still providing sufficient attachment to skin 132.

For a given needle length and for a given channel length, the desired delivery depth, ID, is achieved by controlling the depth of skin depression, D, that remains following insertion of microneedle 142. The depth of skin depression, D, that occurs during microneedle insertion for a particular delivery device is a function of the physical properties of the skin, the sharpness of tip 356 of microneedle 142 and the force supplied by torsion rod 106. As will be explained in more detail below, in one embodiment, delivery device may include a tissue support structure that engages skin 132 to resist the downward depression and/or surface deformation caused by microneedle 142. In this embodiment, the depth of skin depression, D, is also a function of the amount of depression or deformation resistance afforded by the tissue support structure.

Skin depression D decreases as the sharpness of tip 356 increases and width of the needle decreases. Skin depression D also decreases as the force supplied to microneedle array 134 by the microneedle actuator (e.g., torsion rod 106) increases and as the velocity of tips 356 at insertion increases. Thus, for a given tip sharpness and needle length, the microneedle actuator (e.g., torsion rod 106) may be selected to deliver sufficient force to substantially reduce or to minimize skin depression. In one embodiment, the force delivered by the microneedle actuator may be selected to be above a threshold above which skin depression D no longer substantially decreases as a function of the force supplied by the microneedle actuator.

In one embodiment, the sharpness of tip 356 is selected to reduce skin depression D. In another embodiment, the forced supplied by torsion rod 106 is selected to reduce skin depression D. In one embodiment, the sharpness of tip 356 and/or the needle length of microneedles 142 may be determined primarily by the selection of a particular manufacturing technique or by selection of a particular microneedle material. In this embodiment, reduction of skin depression may be accomplished primarily by selecting the force delivered by the microneedle actuator.

Accordingly to various embodiments, the length of the portion of microneedle 142 that extends below the lower surface of adhesive layer 22 is between 0.85 mm and 1.1 mm, preferably between 0.9 mm and 1.05 mm, and more preferably between 0.95 mm and 1 mm. In one preferred embodiment, the length of the portion of microneedle 142 that extends below the lower surface of adhesive layer 22 may be 1 mm, and in another preferred embodiment, the length of the portion of microneedle 142 that extends below the lower surface of adhesive layer 22 may be 0.95 mm. In various embodiments, the radius of curvature of tip 356 (which is a measurement of tip sharpness) may be 17 μm plus or minus 8 μm. In one embodiment, the energy stored in the microneedle actuator (e.g., torsion rod 106) is between 0.015 and 0.025 J, preferably between 0.018 and 0.022 J and even more preferably between 0.019 and 0.021 J. In one preferred embodiment, the energy stored in the microneedle actuator is 0.02 J.

As noted above, reduction of skin depression D may be accomplished by providing a drug delivery device with a tissue support structure that engages skin 132 to resist the downward depression and/or surface deformation caused by microneedle 142. In the embodiment shown, the tissue support structure includes at least one channel, shown as channels 116 formed through bottom wall 61 and adhesive layer 22, a tensile membrane or rigid wall or backing, shown as, but not limited to, the portion of the rigid bottom wall 61 positioned beneath microneedle array 134, and an engagement element, shown as, but not limited to, the portion of the adhesive layer 22 adjacent to channels 116.

Referring to FIG. 13, in one embodiment, the portion of bottom wall 61 below microneedle array 134 forms a tensile membrane or rigid layer or backing to which adhesive layer 22 is attached. Further, in the embodiment shown in FIG. 13, channels 116 are cylindrical channels (e.g., shaped to have a circular cross section) having a substantially constant diameter along the height of the channel. Further, in the embodiment shown, the diameters of channels 116 are substantially the same as the diameter of the base of the microneedles 142.

In the embodiment shown in FIG. 13, the portion of adhesive layer 22 surrounding and adjacent to channel 116 acts as a support structure by resisting depression and/or surface deformation of the skin caused by microneedle 142. The attachment or bond between adhesive layer 22 and skin 132 resists or prevents the downward depression or deformation of skin 132 caused by the downward movement of microneedles 142. In one embodiment, the bond between adhesive layer 22 and skin 132 exerts reaction forces on the skin perpendicular to and in the direction opposite to the movement of microneedle array 134 to resist deformation of the skin. Because adhesive layer 22 is adhered to the outer surface of skin 132 at the periphery of channels 116, adhesive layer 22 tends to maintain the position of the outer surface of skin 132 below channel 116 more precisely than if adhesive layer 22 were not present. In one embodiment, adhesive layer 22 attaches to or anchors the portion of the outer surface of skin 132 adjacent to channel 116 at a fixation point that skin 132 pulls against as the microneedle urges the skin inward and downward away from adhesive layer 22. Adhesive layer 22 geometrically increases the tensile membrane stiffness of the portion of skin 132 below channel 116, and thus, facilitates penetration of skin 132 by microneedle 142. The increased tensile stiffness results in a decrease in compliance of the portion of the skin below the microneedle facilitating piercing of the skin by the microneedle. In one embodiment, the bond between adhesive layer 22 and the skin adjacent to channels 116 tends to pull skin 132 up towards adhesive layer 22 following puncture thereby decreasing the amount of skin depression D that remains following microneedle insertion. In one embodiment, channels 116 surround or encircle microneedle 142 at the point of contact between the tip of microneedle 142 and skin 132, and thus, adhesive layer 22 is adhered to skin 132 adjacent to the entire outer surfaces of microneedles 142. In the case of channels 116, adhesive layer 22 completely surrounds or encircles each microneedle 142 as microneedle 142 is brought into contact with the skin. According to various exemplary embodiments, the diameter of channel 116 is between 1.0 mm and 1.5 mm, preferably is between 1.20 mm and 1.35 mm, and even more preferably is between 1.25 mm and 1.30 mm. In one preferred embodiment, the diameter of channel 116 is 1.27 mm.

Bottom wall 61 provides a tensile membrane or rigid support or anchor for adhesive layer 22 to pull on as adhesive layer 22 acts to resist or prevent the inward and downward depression and/or deformation of skin 132. The effectiveness of adhesive layer 22 as part of a support structure is increased as the strength of the adherence between adhesive layer 22 and the outer surface of skin 132 is increased. The effectiveness of adhesive layer 22 as part of a support structure is also increased as the edge of the adhesive layer at channel 116 is brought closer to shaft 160 of microneedle 142. Thus, the cylindrical channel 116 has a diameter minimized to match the diameter of the base of microneedle 142. In another embodiment, holes 114 in bottom wall 61 and holes 28 in adhesive layer 22 have tapered sidewalls such that the holes have a diameter that decreases in the direction toward the outer surface of adhesive layer 22 forming generally cone-shaped channels 162 having tapered sidewalls. In this embodiment, the diameters of channels 162 at the point of contact between adhesive layer 22 and skin 132 are less than in the case of the cylindrical channels. Thus, tapered channel 162 brings the edge of adhesive layer 22 at channel 162 closer to the point of contact between the tip of microneedle 142 and skin 132 than the cylindrical channels 116.

While the tissue support structure embodiments discussed herein include a layer of adhesive to adhere to the skin to provide support to and to resist inward and downward depression or deformation of the skin surface caused by contact with the microneedle, other skin engagement elements may be used that resist the skin deformation and/or depression. For example in one embodiment, the lower surface of bottom wall 61 below microneedle array 134 may include hook structures to engage the skin adjacent to channels 116 to resist skin surface depression or deformation. In another embodiment, the lower surface of bottom wall 61 below microneedle array 134 may include clamp or pinch structures to engage the skin adjacent to channels 116 to resist skin surface depression or deformation.

Skin depression D may be reduced via a tissue support structure as discussed above. In one embodiment of a drug delivery device 16 including a tissue support structure, needle length, tip sharpness and the force delivered by the microneedle actuator may be less than would otherwise be needed. In one embodiment, needle length, sharpness of tip 356 and the force generated by a microneedle actuator (e.g., by selecting spring materials, spring configurations, etc.), are selected to deliver tip 356 to a desired depth. In another embodiment, delivery device 16 includes a support structure that resists deformation of skin 132 caused by microneedle 142, and needle length, sharpness of tip 356 and the force generated by the microneedle actuator (e.g., torsion rod 106) are selected to deliver tip 356 to a desired depth. Further, the amount of the decrease in skin depression D caused by the tissue support structure may be selected such that tip 356 of microneedle 142 is delivered to a predetermined or desired depth within skin 132. In one embodiment, tip sharpness and the actuator may be configured such that tip 356 of the microneedle passes through the outer layer of the skin upon activation, and the needle length is limited such that the tip does not extend past a desired depth within the skin of the subject. In one embodiment, the desired depth is selected such that tip 356 of microneedle 142 is delivered to the papillary dermis.

Further modifications and alternative embodiments of various aspects of the invention will be apparent to those skilled in the art in view of this description. Accordingly, this description is to be construed as illustrative only. The construction and arrangements of the drug delivery device assembly and the drug delivery device, as shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present invention.

What is claimed is:

1. A device for delivering a drug into the skin of a subject, the device comprising:
a drug reservoir;
a microneedle having a tip, a length, and a tip sharpness, the microneedle coupled to the reservoir; and
a microneedle actuator coupled to the microneedle configured to drive the microneedle into the skin of the subject upon activation;
wherein the tip sharpness and the actuator allow the microneedle to pass through an outer layer of the skin upon activation, and the length is limited such that the tip does not extend past a desired depth below the surface of the skin of the subject, wherein the desired depth is located in the papillary dermis or the reticular dermis.

2. The device of claim 1, wherein the outer layer of the skin is the epidermis and the desired depth is located in the upper half of the reticular dermis.

3. The device of claim 2, wherein the drug is delivered to the subject following activation via the microneedle.

4. The device of claim 2, wherein the microneedle is a hollow microneedle, and further wherein the drug is a liquid drug and is delivered to the subject following activation via the hollow microneedle.

5. The device of claim 1, wherein the device is configured to deliver the drug to the skin of the upper arm of the subject and wherein the desired depth is 100 micrometers to 2 millimeters below the outer surface of the skin.

6. The device of claim 1, wherein the device is configured to deliver the drug to the skin of the abdomen of the subject and wherein the desired depth is 100 micrometers to 1.9 millimeters below the outer surface of the skin.

7. The device of claim 1, further comprising an engagement element configured to adhere to the skin of the subject such that the engagement element resists deformation of the skin surface caused by the microneedle during activation.

8. The device of claim 7, wherein the engagement element comprises an adhesive material and wherein the adhesive material is configured to form a nonpermanent bond to the skin of the subject, the bond being of sufficient strength to resist the deformation of the skin surface caused by the microneedle during activation.

9. The device of claim 8, further comprising a tensile membrane having an upper surface and a lower surface, wherein the adhesive material is coupled to the lower surface of the tensile membrane.

10. The device of claim 9, wherein the adhesive material includes a first hole and the tensile membrane includes a second hole aligned with the first hole, wherein the first and second holes define a channel, the channel having a first end and a second end, the channel in axial alignment with the microneedle, wherein at least the tip extends past the second end of the channel following activation.

11. A drug delivery device for delivering a liquid drug into the skin of a subject, the device comprising:
a drug reservoir storing a dose of the liquid drug;
a conduit coupled to the drug reservoir; and
a hollow microneedle having a tip, a length and a tip sharpness, the hollow microneedle coupled to the conduit, wherein the conduit provides fluid communication between the drug reservoir and the hollow microneedle, such that the drug is permitted to flow from the drug reservoir through the conduit and through the hollow microneedle to the skin of the subject;
a microneedle actuator coupled to the hollow microneedle and configured to drive the hollow microneedle into the skin of the subject upon activation; and
an engagement element configured to adhere to the skin of the subject such that the engagement element resists deformation of the skin surface caused by the hollow microneedle during activation;
wherein at least one of the tip sharpness, the actuator and the engagement element is configured to reduce deformation of the skin surface of the subject caused by the hollow microneedle following activation, and further wherein the microneedle length allows the tip of the hollow microneedle to be delivered to the papillary dermis or reticular dermis of the subject.

12. The device of claim 11, wherein the liquid drug is delivered to the papillary dermis or to the upper half of the reticular dermis of the subject following activation.

13. The device of claim 11, wherein the engagement element comprises an adhesive material, wherein the adhesive material is configured to form a nonpermanent bond to the skin of the subject, the bond being of sufficient strength to resist the deformation of the skin surface caused by the hollow microneedle during activation.

14. A method of delivering a drug to the skin of a subject, the method comprising:
providing a drug delivery device comprising:
a drug reservoir;
a microneedle having a tip, a length and a tip sharpness, the microneedle coupled to the reservoir; and
a microneedle actuator coupled to the microneedle configured to drive the microneedle into the skin of the subject upon activation;
selecting at least one of the length, the tip sharpness and the microneedle actuator to allow the tip to be delivered to a desired depth below the surface of the skin of the subject, wherein the desired depth is located in the papillary dermis or the reticular dermis;
activating the microneedle actuator to insert the microneedle to the desired depth within the skin of the subject; and
delivering the drug to the skin of the subject via the microneedle.

15. The method of claim 14, wherein at least one of the length, the tip sharpness and the microneedle actuator is selected to allow the tip of the microneedle to be delivered to the upper half of the reticular dermis of the subject.

16. The method of claim 15, wherein the drug is delivered to the papillary dermis or the reticular dermis of the subject.

17. The method of claim 14, further comprising attaching the drug delivery device to the outer surface of the skin of the subject.

18. The method of claim 17, wherein the drug delivery device is attached to the skin of the upper arm or abdomen of the subject, and at least one of the length, the tip sharpness and the microneedle actuator is selected to allow the tip of the microneedle to be delivered to a depth of 100 micrometers to 2 millimeters below the outer surface of the skin.

19. The method of claim 14, wherein the drug delivery device further comprises an engagement element configured to adhere to the skin of the subject such that the engagement element resists deformation of the skin surface caused by the microneedle during activation.

20. The method of claim 14, wherein the microneedle is a hollow microneedle and the drug is a liquid drug, and further wherein the delivering step includes delivering the drug to the papillary dermis or upper half of the reticular dermis of the subject via the hollow microneedle.
